(19) United States
(12) Patent Application Publication
(10) Pub. No.: US 2025/0009627 A1
(43) Pub. Date: Jan. 9, 2025

(54) THERAPEUTIC AND NON-THERAPEUTIC USES OF A PERSONAL CLEANSING COMPOSITION AND METHODS OF TREATMENT

(71) Applicant: The Procter & Gamble Company, Cincinnati, OH (US)

(72) Inventors: Rui Li, Singapore (SG); Edward Dewey Smith, III, Mason, OH (US); Myriam Rubecca Rodrigues, Singapore (SG); Randall Thomas Reilman, Cincinnati, OH (US); Karl Shiqing Wei, Mason, OH (US); Olubolaji Akintelure, Mason, OH (US)

(21) Appl. No.: 18/754,995

(22) Filed: Jun. 26, 2024

Related U.S. Application Data
(60) Provisional application No. 63/510,519, filed on Jun. 27, 2023.

(51) Int. Cl. A61K 8/46; A61K 9/00; A61K 31/255; A61P 17/10; A61Q 15/00

(52) U.S. Cl. CPC A61K 8/463; A61K 9/0014; A61K 31/255; A61P 17/10; A61Q 15/00

(57) ABSTRACT

A method of inhibiting or killing the growth of bacteria onto human skin by topically applying onto human skin an effective amount of a personal cleansing composition. The bacteria is selected from Escherichia coli at a pH up to or equal to about 4 in solution, Cutibacterium acnes, and/or Corynebacterium minutissimum. The personal cleansing composition includes a salt of an alkenyl sulfate ester having an alkenyl chain. The alkenyl chain includes from 18 to 20 carbon atoms, or an isomer thereof, or mixtures thereof.

### FIELD OF THE INVENTION

The present application generally relates to therapeutic and non-therapeutic uses of a personal cleansing composition; and methods of treatments relating thereto. The personal cleansing composition comprises a salt of an alkenyl sulfate ester having an alkenyl chain, wherein the alkenyl chain comprises from 18 to 20 carbon atoms.

### BACKGROUND OF THE INVENTION

Human health is impacted by many microbial entities or microbials such as germs, bacteria, fungi, yeasts, molds, viruses and the like. For example, infection by microbial entities or microbials including various viruses and bacteria cause a wide variety of sicknesses and ailments. To reduce such infections, people frequently wash their skin with a personal cleansing composition. Personal cleansing compositions have traditionally been marketed in a variety of forms such as bar soaps, creams, lotions, and gels.

Washing with a personal cleansing composition, such as an antimicrobial soap can remove dirt and germs from skin. Removal of the viruses and bacteria is due to the surfactant behavior of the soap and the mechanical action of the wash procedure. Antibacterial soaps typically include soaps in combination with one or more actives, for example, antimicrobial agents; which can be in the form of a bar of soap. When the skin is washed with an antimicrobial soap, such as a bar soap, the surfactant of the soap typically removes most of the microbial entities or microbials on the skin, while the antimicrobial agent deposits at least in part onto the skin to provide residual protection against subsequent invasion.

Bacteria found on the skin can be divided into two groups: resident and transient bacteria. Resident bacteria are gram-positive bacteria which are established as permanent microcolonies on the surface and outermost layers of the skin and play a relevant, helpful role in preventing the colonization of other, more harmful bacteria and fungi.

Transient bacteria are bacteria which are not part of the normal resident flora of the skin, but can be deposited when airborne contaminated material lands on the skin or when contaminated material is brought into physical contact with it. Transient bacteria are typically divided into two subclasses: Gram-positive and gram-negative bacteria. Gram-positive bacteria include pathogens such as Staphylococcus aureus. Gram-negative bacteria include pathogens such as Escherichia coli. Gram-negative bacteria are generally distinguished from gram-positive by an additional protective cell membrane which generally results in the gram-negative bacteria being less susceptible to topical antibacterial actives.

Antimicrobial cleansing products have been marketed in a variety of forms for some time. Forms include deodorant soaps, antibacterial soaps, and surgical disinfectants. These traditional rinse-off antimicrobial cleansing compositions have been formulated to provide bacteria reduction during washing.

A personal cleansing composition comprising water-soluble metallo surfactants such as sodium oleyl sulfate with or without lanthanum has been developed, see for instance EP 0 018 019 A1.

Corynebacterium minutissimum is part of the Corynebacterium genus that are the primary causal agents of axillary malodors with the key malodor substrates believed to originate from the apocrine gland

There is still a need to formulate a personal cleansing composition which could be used for reducing axillary malodors.

Cutibacterium (formerly Propionibacterium) acnes is known to be associated with skin acne conditions, see for instance US 2011/054039 A1.

As such, it is still desirable to provide a personal cleansing composition having anti-acne benefits.

Some of the antimicrobial cleansing compositions, especially the surgical disinfectants, utilize prominent levels of alcohol and/or harsh surfactants which have been shown to dry out and irritate skin tissues. Ideal personal cleansers should gently cleanse the skin, cause little or no irritation, and not leave the skin overly dry after frequent use and preferably should provide a moisturizing benefit to the skin.

Typically, the personal cleansing composition should satisfy a number of criteria to be acceptable to consumers. These criteria include cleansing effectiveness, skin feel, mildness to skin and ocular mucosae, and lather volume. Ideal personal cleansers should gently cleanse the skin, cause little or no irritation, and should not leave the skin overly dry after frequent use.

Hence, there is also a need to formulate a personal cleansing composition which provides mildness to the skin and with acceptable cleansing, lather or foaming properties.

### SUMMARY OF THE INVENTION

A personal cleansing composition is provided for use in a method of inhibiting or killing the growth of bacteria onto human skin, wherein the bacteria is selected from the group consisting of Escherichia coli at a pH up to or equal to about 4 in solution, Cutibacterium acnes, Corynebacterium minutissimum; wherein the personal cleansing composition comprises a salt of an alkenyl sulfate ester having an alkenyl chain, wherein the alkenyl chain comprises from 18 to 20 carbon atoms, preferably 18 carbon atoms, or an isomer thereof, or mixtures thereof.

A personal cleansing composition is provided for use in a method of treating acne and eradicating Cutibacterium acnes onto a human skin; preferably onto a facial skin; wherein the personal cleansing composition comprises a salt of an alkenyl sulfate ester having an alkenyl chain, wherein the alkenyl chain comprises from 18 to 20 carbon atoms, preferably 18 carbon atoms, or an isomer thereof, or mixtures thereof.

A personal cleansing composition is provided for use in a method of treating axillary malodors and killing the growth of Corynebacterium minutissimum onto human skin; wherein the personal cleansing composition comprises a salt of an alkenyl sulfate ester having an alkenyl chain, wherein the alkenyl chain comprises from 18 to 20 carbon atoms, preferably 18 carbon atoms, or an isomer thereof, or mixtures thereof.

A non-therapeutic use of a personal cleansing composition is provided for cleaning skin with mildness; preferably compared to a control composition comprising 10 wt. % of sodium lauryl sulfate, 0.05 wt. % of kathon CG, q.s. citric acid at pH of 5.5±0.2 in q.s. water; wherein the personal cleansing composition comprises a salt of an alkenyl sulfate ester having an alkenyl chain, wherein the alkenyl chain comprises from 18 to 20 carbon atoms, preferably 18 carbon atoms, or an isomer thereof, or mixtures thereof.

A non-therapeutic use of a personal cleansing composition is provided for preventing dryness of the skin; wherein the personal cleansing composition comprises a salt of an alkenyl sulfate ester having an alkenyl chain, wherein the alkenyl chain comprises from 18 to 20 carbon atoms, preferably 18 carbon atoms, or an isomer thereof, or mixtures thereof.

### DETAILED DESCRIPTION OF THE INVENTION

### Definitions of Terms

In this document, the following definitions apply unless specifically stated otherwise.

All percentages are by weight (w/w) of the composition, unless otherwise specified. “% wt.” means percentage by weight. References to ‘parts’ e.g. a mixture of 1 part X and 3 parts Y, is a ratio by weight. All ratios or percentages are weight ratios or weight percentages unless specifically stated otherwise.

An “active composition” is the composition absent water, and an “active ingredient” is the ingredient absent its water.

“QS” or “QSP” means sufficient quantity for 100% or for 100 g. +/−indicates the standard deviation. All ranges are inclusive and combinable. The number of significant digits conveys neither a limitation on the indicated amounts nor on the accuracy of the measurements. All numerical amounts are understood to be modified by the word “about”.

All measurements are understood to be made at 25° C. and at ambient conditions, where “ambient conditions” means at 1 atmosphere (atm) of pressure and at 65% relative humidity, unless otherwise stated. “Relative humidity” refers to the ratio (stated as a percent) of the moisture content of air compared to the saturated moisture level at the same temperature and pressure. Relative humidity can be measured with a hygrometer, in particular with a probe hygrometer from VWR® International.

Herein “min” means “minute” or “minutes”. Herein “mol” means mole. Herein “g” following a number means “gram” or “grams”. “Ex.” means “example”. All amounts as they pertain to listed ingredients are based on the active level and do not include carriers or by-products that may be included in commercially available materials.

Herein, “comprising” means that other steps and other ingredients can be in addition. “Comprising” encompasses the terms “consisting of” and “consisting essentially of”. The compositions, methods, uses, and processes described herein can comprise, consist of, and consist essentially of the elements and limitations described herein, as well as any of the additional or optional ingredients, components, steps, or limitations described herein. Embodiments and aspects described herein may comprise or be combinable with elements, features or components of other embodiments and/or aspects despite not being expressly exemplified in combination, unless an incompatibility is stated.

As used herein, the articles including “a” and “an” when used in a claim, are understood to mean “one or more” of what is claimed or described.

The terms “include,” “includes,” and “including,” as used herein are meant to be non-limiting.

Where amount ranges are given, these are to be understood as being the total amount of said ingredient in the composition, or where more than one species fall within the scope of the ingredient definition, the total amount of all ingredients fitting that definition, in the composition.

For example, if the composition comprises from 1% to 5% fatty alcohol, then a composition comprising 2% stearyl alcohol and 1% cetyl alcohol and no other fatty alcohol, would fall within this scope.

The amount of each particular ingredient or mixtures thereof described hereinafter can account for up to 100% (or 100%) of the total amount of the ingredient(s) in the composition.

The term “free of” as used herein means that the composition comprises 0% of an ingredient by weight of the composition, thus no detectable amount of the stated ingredient.

The term “substantially free of” as used herein means less than about 1%, less than about 0.8%, less than about 0.5%, less than about 0.3%, or less than an immaterial amount of by weight of the composition.

Herein “Comp. Ex.” or “C. Ex.” means comparative example; and “Ex.” means example.

The term “molecular weight” or “M.Wt.” as used herein refers to the weight average molecular weight unless otherwise stated. The weight average molecular weight can be measured by gel permeation chromatography (“GPC”).

The term “personal cleansing composition” as used herein refers to compositions intended for topical application to the skin for cleansing.

The term “mixtures” as used herein is meant to include a simple combination of materials and any compounds that may result from their combination.

The term “room temperature” refers to a temperature of 25° C.

The term “rinse-off” as used herein means the intended product usage includes application to skin followed by rinsing and/or wiping the product from the skin within a few seconds to minutes of the application step. The product is generally applied and rinsed in the same usage event, for example, a shower or washing one's hands.

The term “derivative” as used herein refers to structures which are not shown but which one skilled in the art would understand are variations of the basic compound.

The term “apply” or “application,” as used in reference to a composition, means to apply or spread the compositions described herein onto the skin.

The term “Biodegradable Test” as used herein means the test as described in the Test Method Section herein in which the test material is suspended in a phosphate buffered media containing an activated sludge inoculum and the formation of carbon dioxide measured via a closed-circuit respirometer. The test material is the sole carbon and energy source and under aerobic conditions microorganisms metabolize organic substances producing CO₂ as the ultimate product.

The term “dermatologically acceptable” means that the compositions or components described are suitable for use in contact with human skin tissue without undue toxicity, incompatibility, instability, allergic response, and the like.

The term “cosmetically acceptable,” as used herein, means that the compositions, formulations or components described are suitable for use in contact with human keratinous tissue without undue toxicity, incompatibility, instability, allergic response, and the like. All compositions described herein which have the purpose of being directly applied to keratinous tissue are limited to those being cosmetically acceptable.

The methods as disclosed herein are cosmetic methods or non-therapeutic methods unless specifically stated otherwise.

The new uses of personal cleansing compositions, methods of uses of the products, the structures and the respective compositions as described in the Summary or as described hereinbelow are for fulfilling the technical effects or goals as set out herein. These objects and other advantages as may be apparent to those skilled in the art can be achieved through the new uses of personal cleansing compositions, methods of uses of the products, the structures and the respective compositions as described herein.

### Benefits

A salt of an alkenyl sulfate ester having an alkenyl chain, as defined herein, especially but not limited to sodium oleyl sulfate showed superior antibacterial performance against gram-positive bacteria including C. minutissimum (odor relevant) and C. acnes (acne relevant); also on Gram-negative bacteria Escherichia coli at a pH up to or equal to 4 in solution.

A personal cleansing composition is provided for use in a method of inhibiting or killing the growth of bacteria onto human skin, wherein the bacteria is selected from the group consisting of Escherichia coli at a pH up to or equal to 4 in solution, Cutibacterium acnes, Corynebacterium minutissimum; wherein the personal cleansing composition comprises a salt of an alkenyl sulfate ester having an alkenyl chain, wherein the alkenyl chain comprises from 18 to 20 carbon atoms, preferably 18 carbon atoms, or an isomer thereof, or mixtures thereof.

The MIC result of sodium oleyl sulfate against gram-positive bacteria has been found to be in the range of potent biocides like benzalkonium chloride.

Hence, a salt of an alkenyl sulfate ester having an alkenyl chain, wherein the alkenyl chain comprises from 18 to 20 carbon atoms; preferably 18 carbon atoms, or an isomer thereof, or mixtures thereof; such as sodium oleyl sulfate or a personal cleansing composition as set out herein can be provided in a method of treating acne and eradicating Cutibacterium acnes onto a human skin; preferably onto a facial skin; or in a method of treating axillary malodors and killing the growth of Corynebacterium minutissimum onto human skin.

Surprisingly, when setting the pH up to about 4, it was possible to observe some bactericidal activity of an alkenyl sulfate ester having an alkenyl chain, as defined herein, especially but not limited to sodium oleyl sulfate against E. coli in solution.

A salt of an alkenyl sulfate ester having an alkenyl chain, as defined herein, especially but not limited to sodium oleyl sulfate is also compatible with commonly used surfactants like sodium lauryl sulfate, or sodium laureth(n) sulfate, hereinafter SLEnS, wherein n which is the average moles of ethoxylation being between 1 and 3; preferably 1 or 3, and/or a betaine such as cocamidopropyl betaine and showed good formula feasibility in terms of foaming properties.

A salt of an alkenyl sulfate ester having an alkenyl chain, as defined herein, especially but not limited to sodium oleyl sulfate has been found to be milder to skin than sodium lauryl sulfate due to its unsaturated chain length.

A salt of an alkenyl sulfate ester having an alkenyl chain, as defined herein, especially but not limited to sodium oleyl sulfate (SOS) is considered “biodegradable” according to the Biodegradability Test Method that is based on the Organization for Economic Co-operation and Development (OECD) 301B CO₂ evolution biodegradation test method. The test method screens chemicals for ready biodegradability in an aerobic aqueous medium (see Example section).

### Therapeutic and Non-Therapeutic Uses

All the limitations and aspects as disclosed herein for the personal cleansing composition, or the salt of an alkenyl sulfate might apply herein below.

It has been found that the salt of the alkenyl sulfate ester as set out herein and thus the personal cleansing composition thereto can inhibit or kill specific bacteria for providing long-lasting protection for the human body against transient pathogenic microorganisms.

As shown in more details below, minimum inhibitory concentration (MIC) testing and Kill Rate Testing have been carried out for the salt of the alkenyl sulfate ester as described herein.

A salt of an alkenyl sulfate ester having an alkenyl chain, as defined herein, especially but not limited to sodium oleyl sulfate showed superior antibacterial performance against gram-positive bacteria including C. minutissimum (odor relevant) and C. acnes (acne relevant); also on Gram-negative bacteria Escherichia coli at a pH up to or equal to about 4 in solution.

A personal cleansing composition is provided for use in a method of inhibiting or killing the growth of bacteria onto human skin, wherein the bacteria is selected from the group consisting of Escherichia coli at a pH up to or equal to about 4 in solution, Cutibacterium acnes, Corynebacterium minutissimum; wherein the personal cleansing composition comprises a salt of an alkenyl sulfate ester having an alkenyl chain, wherein the alkenyl chain comprises from 18 to 20 carbon atoms, preferably 18 carbon atoms, or an isomer thereof, or mixtures thereof.

The minimum inhibitory concentration results have shown that the salt of the alkenyl sulfate ester or the personal cleansing composition as set out herein can inhibit the growth of Escherichia coli at a pH up to or equal to about 4 in solution and other gram-positive bacteria such as Corynebacterium minutissimum and Cutibacterium acnes. The minimum inhibitory concentration results of the salt of the alkenyl sulfate ester or the personal cleansing composition as set out herein are in the range of potent biocides such as benzalkonium chloride.

Hence, a personal cleansing composition as set out herein is provided for use in delivering an antimicrobial benefit on a topical surface of a human body. The topical surface of the human body may be a keratinous tissue surface. A keratinous tissue refers to keratin-containing layers positioned as the outermost protective covering of mammals which includes, but is not limited to, skin, hair, nails, or cuticles.

The topical surface of the human body may be preferably the human skin surface, or scalp surface.

It has been also found that the salt of the alkenyl sulfate ester as set out herein can demonstrate relatively strong bacterial inhibition effect against Corynebacterium minutissimum, and Cutibacterium acnes.

Corynebacterium minutissimum is part of the Corynebacterium genus that are the primary causal agents of axillary malodors with the key malodor substrates believed to originate from the apocrine gland. Thus, the salt of the alkenyl sulfate ester and the personal cleansing composition as set out herein can deliver anti-malodor benefits.

Also, the salt of the alkenyl sulfate ester as set out herein may be provided for use in a method of eradicating Cutibacterium acnes onto a human skin; preferably onto a facial skin.

The personal cleansing composition as set out herein is provided for use in a method of eradicating Cutibacterium acnes onto a human skin; preferably onto a facial skin.

Cutibacterium (formerly Propionibacterium) acnes is known to be associated with skin acne conditions. Thus, the salt of the alkenyl sulfate ester as set out herein can deliver anti-acne benefits.

It has been found that the salt of the alkenyl sulfate ester as set out herein did not exhibit an acceptable MIC for gram-negative bacteria such as Escherichia coli. The MIC testing has been carried out at a pH of about 7.2.

However, in solution and at a certain acidic pH up to about 4, the salt of the alkenyl sulfate ester as set out herein could kill the gram-negative bacteria Escherichia coli.

Hence, the salt of the alkenyl sulfate ester as set out herein may be provided for use in a method of killing the growth of Escherichia coli under a pH up to or equal to about 4 in solution.

The personal cleansing composition as set out herein is provided for use in a method of killing the growth of Escherichia coli under a pH up to or equal to about 4 in solution.

Non-therapeutic or cosmetic uses have been also found. Non-therapeutic use of the personal cleansing composition as set out herein is provided for cleaning skin; preferably with mildness. The use of the personal cleansing composition as set out herein for cleaning skin with mildness is compared to a control composition. The control composition includes or consists of about 10 wt. % of sodium lauryl sulfate, about 0.05 wt. % of kathon CG (a combination of methylchloroisothiazolinone and methylisothiazolinone), q.s. citric acid at pH of about 5.5±0.2 in q.s. water.

The mini-immersion corneometer results as shown below have indicated that a personal cleansing composition comprising sodium oleyl sulfate as a salt of an alkenyl sulfate ester having an alkenyl chain, wherein the alkenyl chain comprises from 18 to 20 carbon atoms, preferably 18 carbon atoms as set out herein is significantly milder than a control composition including sodium lauryl sulfate.

Non-therapeutic use of the personal cleansing composition as set out herein for its mildness towards the skin; preferably for preventing dryness of the skin.

Non-therapeutic use of a salt of an alkenyl sulfate ester having an alkenyl chain, wherein the alkenyl chain comprises from 18 to 20 carbon atoms, preferably 18 carbon atoms, or an isomer thereof, or mixtures thereof; preferably sodium oleyl sulfate in a personal cleansing composition to increase the mildness to the skin of the composition. By increasing the mildness to the skin of the composition, it is intended meaning preventing skin tightness, skin roughness and/or skin irritation.

As seen above, the salt of an alkenyl sulfate ester having an alkenyl chain, wherein the alkenyl chain comprises from 18 to 20 carbon atoms preferably 18 carbon atoms as set out herein; preferably sodium oleyl sulfate can inhibit Corynebacterium minutissimum responsible of axillary malodors.

Hence, non-therapeutical use of a salt of an alkenyl sulfate ester having an alkenyl chain, wherein the alkenyl chain comprises from 18 to 20 carbon atoms, preferably 18 carbon atoms as set out herein; preferably sodium oleyl sulfate as a malodor counteracting agent on products selected from the group consisting of consumer products, household products, cosmetic and personal care products, products for use on the human body, products applied to the human skin, and perfumed consumer goods.

The salt of the alkenyl sulfate ester having an alkenyl chain, wherein the alkenyl chain comprises from 18 to 20 carbon atoms, preferably 18 carbon atoms as set out herein; preferably sodium oleyl sulfate may be used as a malodor counteracting agent in a personal cleansing composition onto the axillary (underarm) skin surface.

Alternatively, the salt of the alkenyl sulfate ester having an alkenyl chain, wherein the alkenyl chain comprises from 18 to 20 carbon atoms, preferably 18 carbon atoms as set out herein; preferably sodium oleyl sulfate may be used as a preservative in a personal cleansing composition.

In the personal cleansing composition, the salt of the alkenyl sulfate ester as used herein; preferably sodium oleyl sulfate may be present in an amount ranging from about 0.01-5% by weight of the composition; preferably in an amount ranging from about 0.02-3% by weight of the composition; more preferably in an amount ranging from about 0.05-1.5% by weight of the composition; even more preferably in an amount ranging from about 0.1-1% by weight of the composition; most preferably in an amount ranging from about 0.2-0.5% by weight of the composition.

Non-therapeutic use of the personal cleansing composition as set out herein for providing a cleansing benefit.

Non-therapeutic use of the personal cleansing composition as set out herein for improving hand hygiene.

The personal cleansing composition, the salt of an alkenyl sulfate and any forms of the composition will be described in more details below.

### Personal Cleansing Composition and Forms

A personal cleansing composition is provided and comprises a salt of an alkenyl sulfate ester having an alkenyl chain, wherein the alkenyl chain comprises from 18 to 20 carbon atoms, preferably 18 atom carbons, or an isomer thereof, or mixtures thereof.

The salt of an alkenyl sulfate ester has the function of an antimicrobial active on specific bacteria as recited herein. Also, the salt of an alkenyl sulfate ester may have the function of an anionic surfactant.

A salt of an alkenyl sulfate ester as disclosed herein comprises a sulfate ester group (OSO₃M) and has the formula (I) as follows:

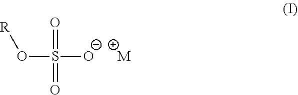

R is an alkenyl chain and the alkenyl chain comprises from 18 to 20 carbon atoms, preferably 18 atom carbons. The alkenyl chain is directly connected to the oxygen of the sulfate ester group (OSO₃M). Hence, the alkenyl chain R only includes carbons and hydrogens; and not any organic function such as an hydroxy group, an ether group, an ester, an amide, or any amino groups within the carbon backbone.

The alkenyl chain R may comprise a straight alkenyl chain. The alkenyl chain R can have one or more double bonds.

Preferably, the alkenyl chain of the salt of the alkenyl sulfate ester may have 18 carbon atoms.

The salt of the alkenyl sulfate ester may be selected from the group consisting of an alkali metal, ammonium, and alkanolamine. The alkali metal may be lithium, sodium or potassium. The alkanolamine may be methylamine, dimethylamine, trimethylamine, ethylamine, diethylamine, or triethylamine.

Preferably, the salt (M) of the alkenyl sulfate ester may be selected from the group consisting of sodium, potassium, triethylamine, and ammonium.

More preferably, the salt (M) of the alkenyl sulfate ester may be selected from the group consisting of sodium and ammonium.

Especially, the salt (M) of the alkenyl sulfate ester is sodium.

The salt of the alkenyl sulfate ester having the alkenyl chain may be present in an amount ranging from about 0.01-50% by weight of the composition; preferably in an amount ranging from about 0.01-30% by weight of the composition; more preferably in an amount ranging from about 0.02-10% by weight of the composition; even more preferably in an amount ranging from about 0.05-5% by weight of the composition; most preferably in an amount ranging from about 0.1-1% by weight of the composition.

Alternatively, the salt of the alkenyl sulfate ester having the alkenyl chain may be present in an amount ranging from about 0.01-5% by weight of the composition; preferably in an amount ranging from about 0.02-3% by weight of the composition; more preferably in an amount ranging from about 0.05-1.5% by weight of the composition; even more preferably in an amount ranging from about 0.1-1% by weight of the composition; most preferably in an amount ranging from about 0.2-0.5% by weight of the composition. This is for example the case when the composition is used as a hand sanitizer.

The salt of the alkenyl sulfate ester having the alkenyl chain may be selected from the group consisting of sodium oleyl sulfate, TEA-oleyl sulfate, ammonium oleyl sulfate, sodium elaidyl sulfate, sodium petroselinyl sulfate, sodium linoleyl sulfate, sodium linolenyl sulfate, sodium gadoleyl sulfate, or an isomer thereof, and mixtures thereof.

The salt of the alkenyl sulfate ester can be prepared from sulfation from the corresponding unsaturated alcohols.

A typical example of unsaturated alcohols whose sulfates can serve as an antimicrobial active in the personal cleansing composition may comprise, in particular oleyl alcohol (cis-9-Octadecen-1-ol with an alkenyl chain of 18 carbon atoms), elaidyl alcohol (trans-9-Octadecenol with an alkenyl chain of 18 carbon atoms), petroselinyl alcohol (cis-6-Octadecen-1-ol with an alkenyl chain of 18 carbon atoms), linoleyl alcohol (cis,cis-9,12-Octadecadien-1-ol with an alkenyl chain of 18 carbon atoms and with two unsaturations), linolenyl alcohol (cis,cis,cis-9,12,15-Octadecatrienol with an alkenyl chain of 18 carbon atoms and with three unsaturations), or gadoleyl alcohol ((Z)-icos-9-en-1-ol with an alkenyl chain of 20 carbon atoms).

Preferably, unsaturated alcohols whose sulfates can serve as an antimicrobial active in the personal cleansing composition may comprise, in particular, oleyl alcohol (cis-9-Octadecen-1-ol with an alkenyl chain of 18 carbon atoms), or gadoleyl alcohol ((Z)-icos-9-en-1-ol with an alkenyl chain of 20 carbon atoms).

More preferably, unsaturated alcohols whose sulfates can serve as an antimicrobial active in the personal cleansing composition may comprise, in particular, oleyl alcohol (cis-9-Octadecen-1-ol with an alkenyl chain of 18 carbon atoms).

Unsaturated alcohols whose sulfates can serve as an antimicrobial active may include at least one double bond; preferably at position 9 (between carbon 9 and carbon 10); more preferably one single double bond at position 9. Unsaturated alcohols whose sulfates can serve as an antimicrobial active may include a cis-isomer; preferably may include essentially a cis-isomer.

The use of oleyl alcohol is thus preferred. The salt of the alkenyl sulfate ester may be selected from the group consisting of sodium oleyl sulfate, TEA-oleyl sulfate, ammonium oleyl sulfate, or an isomer thereof, and mixtures thereof.

Preferably, the salt of the alkenyl sulfate ester may comprise sodium oleyl sulfate or ammonium oleyl sulfate. More preferably, the salt of the alkenyl sulfate ester may be sodium oleyl sulfate.

Sodium oleyl sulfate may be present in an amount ranging from about 0.01-50% by weight of the composition; preferably in an amount ranging from about 0.01-30% by weight of the composition; more preferably in an amount ranging from about 0.02-10% by weight of the composition; even more preferably in an amount ranging from about 0.05-5% by weight of the composition; most preferably in an amount ranging from about 0.1-1% by weight of the composition.

Alternatively, sodium oleyl sulfate may be present in an amount ranging from about 0.01-5% by weight of the composition; preferably in an amount ranging from about 0.02-3% by weight of the composition; more preferably in an amount ranging from about 0.05-1.5% by weight of the composition; even more preferably in an amount ranging from about 0.1-1% by weight of the composition; most preferably in an amount ranging from about 0.2-0.5% by weight of the composition. This is for example the case when the composition is used as a hand sanitizer.

Typically, the salt of the alkenyl sulfate ester as described hereinbefore may have a final active level from about 85 wt. % to about 99.9 wt. %; preferably from 85 wt. % to 90 wt. % as measured by the colorimetric two-phase (water/chloroform) mixed indicator (Dimidium Bromide/Patent Blue VF) titration method using Hyamine 1622 as the cationic titrant (ASTM International standard method D-3049-89 “Standard Test Method for Synthetic Anionic Ingredient by Cationic Titration”).

The salt of the alkenyl sulfate ester as described hereinbefore may comprise a mixture of cis- and trans-isomers.

Preferably, the salt of the alkenyl sulfate ester as described hereinbefore may comprise a cis-isomer; more preferably wherein the salt of the alkenyl sulfate ester as described hereinbefore comprises a mixture of a cis-isomer and a trans-isomer with a weight ratio cis-isomer to trans-isomer from about 8:1 to about 99:1 or from about 8.5:1 to about 95:1 or from about 8.5:1 to about 9:1. Most preferably, the salt of the alkenyl sulfate ester as described hereinbefore may comprise essentially a cis-isomer.

Preferably, sodium oleyl sulfate may comprise a cis-isomer; more preferably wherein sodium oleyl sulfate comprises a mixture of a cis-isomer and a trans-isomer with a weight ratio cis-isomer to trans-isomer from about 8:1 to about 99:1 or from about 8.5:1 to about 95:1 or from about 8.5:1 to about 9:1. Most preferably, sodium oleyl sulfate may comprise essentially a cis-isomer.

The salt of the alkenyl sulfate ester as described hereinbefore may comprise at least one double bond; preferably at position 9 (between carbon 9 and carbon 10); more preferably one single double bond at position 9.

Preferably, sodium oleyl sulfate may comprise one single double bond at position 9.

The personal cleansing composition may be in the form of a topical personal cleansing composition.

The topical personal cleansing composition may be selected from the group consisting of shampoos, creams, lotions, sprays, tonics, gels, paste, mousses, serums, oils, solid or liquid soaps, shower gels, hair conditioning composition, hair moisturizing composition, hair strengthening composition, hair drying composition, hair shaping composition, face lotions, body lotions, skin moisturizing compositions, shaving preparations, hand cleansers, water-less hand sanitizer, facial cleansers, powders, deodorants, anti-dandruff hair care composition, and anti-fungal hair care composition.

Another example of a personal cleansing composition may be a hand sanitizer. A hand sanitizer may be usually a leave on product. A hand sanitizer may comprise, for example, an alcohol, an antibacterial active, a fragrance, surfactant, a colorant, beads, or any combination thereof. Alcohols for use herein can include, for example, ethanol, propanol, or a combination thereof. The alcohol may be present, for example, at a level of 30% to 80%, by weight of a hand sanitizing composition.

The personal cleansing compositions may be in the form of solutions, dispersion, emulsions, foams, and other delivery mechanisms.

The personal cleansing compositions herein may be liquid personal cleansing compositions. Preferably, the liquid personal cleansing composition may be selected from the group consisting of liquid hand washing composition, liquid body washing composition, liquid hair washing composition, and combinations thereof. Preferably, the composition may be a liquid hand washing composition. One example of a composition herein may be a skin cleansing composition. The compositions herein may be single phase or multi-phase.

The personal cleansing composition may be extrudable or dispensable from a single chamber package. The personal cleansing compositions can be in the form of liquid, semi-liquid, cream, lotion or gel, or solid compositions intended for topical application to skin or hair.

Examples of personal cleansing compositions can include but are not limited to shampoo, conditioning shampoo, hair conditioner, body wash, moisturizing body wash, foaming body wash, shower gels, a shower or bath cream, skin cleansers, cleansing milks, hair and body wash, in shower body moisturizer, gel, emulsion, oil, mousse or spray.

The product forms contemplated for purposes of defining the personal cleansing compositions and methods are rinse-off formulations by which it is meant that the product is applied topically to the skin or hair and then subsequently (i.e., within minutes) rinsed away with water, or otherwise wiped off using a substrate or other suitable removal means.

A personal cleansing composition is provided and comprises the salt of an alkenyl sulfate ester as set out herein and a dermatologically acceptable carrier for the personal cleansing composition.

The dermatologically acceptable carrier may be present at a level of from about 50% to about 99%, about 60% to about 98%, about 70% to about 98%, or, alternatively, from about 80% to about 95%, by weight of the composition.

The personal cleansing composition may comprise a dermatologically acceptable carrier. The dermatologically acceptable carrier is any carrier suitable for formulating the salt of the alkenyl sulfate ester into a personal cleansing composition being suitable for application onto skin. The dermatologically acceptable carrier may be selected from either an aqueous medium or an aqueous-alcoholic medium. When the dermatologically acceptable carrier is an aqueous-alcoholic carrier, the dermatologically acceptable carrier may comprise water and an alcohol. An alcohol can advantageously influence the viscosity of a relatively wide spectrum of ingredients of the composition. The alcohol of the personal cleansing composition may be selected from the group consisting of ethanol, isopropanol, propanol, and mixtures thereof.

Inclusion of an appropriate quantity of a dermatologically acceptable carrier can facilitate the formation of the personal cleansing composition having an appropriate liquid viscosity and rheology.

Alternatively, the dermatologically acceptable carrier may be water, or may be a miscible mixture of water and organic solvent. Alternatively, a liquid carrier may be water with minimal or no significant concentrations of organic solvent, except as otherwise incidentally incorporated into the composition as minor ingredients of other essential or optional components. Suitable organic solvents may include water solutions of lower alkyl alcohols and polyhydric alcohols. Useful lower alkyl alcohols include monohydric alcohols having 1 to 6 carbon atoms, such as ethanol and isopropanol. Exemplary polyhydric alcohols include propylene glycol, hexylene glycol, glycerin, and propane diol.

Suitable organic solvents may include monohydric alcohols, dihydric alcohols, polyhydric alcohols, glycerol, glycols, polyalkylene glycols such as polyethylene glycol, and mixtures thereof. Particularly suitable organic solvents may include lower aliphatic alcohols such as ethanol, propanol, butanol, isopropanol; diols such as 1,2-propanediol, 1,3-propanediol, butanediol, pentanediol, hexanediol, heptanediol, octanediol, ethylhexanediol, decanediol; glycerin; water, and mixtures thereof. A composition can comprise, for example, water, a diol, glycerin, or combinations thereof.

When the dermatologically acceptable carrier is an aqueous carrier, the aqueous carrier may consist essentially of water and may be substantially free of alcohol. The personal cleansing composition may comprise a safe and effective amount of a dermatologically acceptable carrier which is water. The personal cleansing composition may comprise from 0.1% to 99%, or from 1% to 98%, or from 10% to 97%, or from 30% to 95% of water by total weight of the composition.

The personal cleansing composition may be substantially free of alcohol, such as volatile alcohols (e.g. ethanol, isopropanol, propanol). When the personal cleansing composition is substantially free of alcohol, the personal cleansing composition can advantageously have a reduced odour. Flammability issues can also be prevented.

The dermatologically acceptable carrier can be in a wide variety of forms. In some instances, the solubility or dispersibility of the salt of the alkenyl sulfate ester or other components of the composition may dictate the form and character of the carrier. Non-limiting examples include simple solutions (e.g., aqueous or anhydrous), dispersions, emulsions, and solid forms (e.g., gels, sticks, flowable solids, or amorphous materials).

In some instances, the dermatologically acceptable carrier may be in the form of an emulsion. The emulsion may have a continuous aqueous phase (e.g., an oil-in-water or water-in-oil-in-water emulsion) or a continuous oil phase (e.g., water-in-oil or oil-in-water-in-oil emulsion). The oil phase may comprise silicone oils, non-silicone oils such as hydrocarbon oils, esters, ethers, and mixtures thereof. The aqueous phase typically comprises water and water-soluble ingredients (e.g. humectants and/or other skin care actives). In some instances, the non-water component of the composition comprises a humectant such as glycerin and/or other polyol(s).

In some instances, the compositions herein may be in the form of an oil-in-water (“O/W”) emulsion that provides a sensorial feel that is light and non-greasy. Suitable O/W emulsions herein may include a continuous aqueous phase of more than 50% by weight of the composition, and the remainder being the dispersed oil phase. The aqueous phase may include 1% to 99% water, based on the weight of the aqueous phase, along with any water soluble and/or water miscible ingredients. In these instances, the dispersed oil phase will typically be present at less than 30% by weight of composition (e.g., 1% to 20%, 2% to 15%, 3% to 12%, 4% to 10%, or even 5% to 8%) to help avoid some of the undesirable feel effects of oily compositions. The oil phase may include one or more volatile and/or non-volatile oils (e.g., botanical oils, silicone oils, and/or hydrocarbon oils). Some nonlimiting examples of oils that may be suitable for use in the present compositions are disclosed in U.S. Pat. No. 9,446,265 and U.S. Publication No. 2015/0196464.

The personal cleansing composition may further comprise sodium lauryl sulfate; preferably from about 0.5% to about 10% of sodium lauryl sulfate by weight of the composition; more preferably from about 1% to about 5% of sodium lauryl sulfate by weight of the composition; most preferably from about 2% to about 5% of sodium lauryl sulfate by weight of the composition. Adding sodium lauryl sulfate to the composition can help for increasing lather volume and foaming properties.

### Optional Ingredients

As can be appreciated, the compositions described herein may include a variety of optional components to tailor the properties and characteristics of the composition. As can be appreciated, suitable optional components are well known and can generally include any components which are physically and chemically compatible with the essential components of the compositions described herein. Optional components should not otherwise unduly impair product stability, aesthetics, or performance. Also, the optional ingredients, when incorporated into the composition, should be suitable for use in contact with human skin tissue without undue toxicity, incompatibility, instability, allergic response, and the like.

Individual concentrations of optional components can generally range from about 0.0001% to about 50%; from about 0.001% to about 20%; or even from about 0.01% to about 10% by weight of the composition. Optional components can be further limited to components which will not impair the clarity of a translucent composition.

Optional components may include, but are not limited to, conditioning agents (including hydrocarbon oils, fatty esters, silicones), cationic polymers, anti-dandruff actives, and chelating agents. Additional suitable optional ingredients include but are not limited to encapsulated and non-encapsulated perfumes or fragrances, colorants, particles, foam boosters, anti-static agents, moisturizing agents, exfoliating agents, skin lightening agents, lubricants, propellants, self-foaming agents, pH adjusting agents and buffers, preservatives, pearlescent agents, opacifiers, sensates, suspending agents, solvents, diluents, anti-oxidants, vitamins, minerals, peptides and peptide derivatives, sugar amines, sunscreens, oil control agents and combinations thereof.

The CTFA Cosmetic Ingredient Handbook, Tenth Edition (published by the Cosmetic, Toiletry, and Fragrance Association, Inc., Washington, D.C.) (2004) (hereinafter “CTFA”), describes a wide variety of nonlimiting materials that can be added to the composition herein.

### Methods of Uses

A method of inhibiting the population of bacteria on an area of need of treatment; preferably wherein the bacteria is Corynebacterium minutissimum, Cutibacterium acnes and/or Escherichia coli at a specific condition, is provided.

A method of inhibiting or killing the growth of bacteria on an area of need of treatment; wherein the bacteria is Escherichia coli at a pH up to or equal to 4, Corynebacterium minutissimum, and/or Cutibacterium acnes, is provided and comprises the step of topically applying onto an area of need of treatment an effective amount of a salt of an alkenyl sulfate ester as described herein, and optionally, removing the composition following its application.

The salt of an alkenyl sulfate ester may have an alkenyl chain, wherein the alkenyl chain comprises from 18 to 20 carbon atoms, preferably 18 carbon atoms as set out herein; preferably sodium oleyl sulfate, and optionally, removing the composition following its application.

A method of killing a bacteria on a skin surface; wherein the bacteria is Escherichia coli at a pH up to or equal to 4, Corynebacterium minutissimum, and/or Cutibacterium acnes, comprising the step of topically applying onto an area of need of treatment an effective amount of a personal cleansing composition as set out herein, and optionally, removing the composition following its application.

An area of need of treatment may be a keratinous tissue surface. A keratinous tissue refers to keratin-containing layers positioned as the outermost protective covering of mammals which includes, but is not limited to, skin, hair, nails, or cuticles.

The area of need of treatment may be preferably the human skin surface, or scalp surface.

A method of controlling or eradicating Cutibacterium acnes from a user's skin is provided and comprises the step of applying a personal cleansing composition as set out herein onto a desired skin surface of a user in need thereof; preferably onto a facial skin surface.

The personal cleansing composition as set out herein can deliver anti-acne benefits. The method of controlling or eradicating Cutibacterium acnes from skin may be provided for reducing or eliminating acne on skin.

A method of controlling or eradicating Corynebacterium minutissimum from an axillary skin surface is provided and comprises the step of applying a personal cleansing composition as set out herein onto the desired skin surface of a user in need thereof; preferably onto the axillary skin surface.

The personal cleansing composition as set out herein can deliver anti-malodor benefits. The method of controlling or eradicating Corynebacterium minutissimum from an axillary skin surface may be provided for minimizing malodors or unpleasant odors caused by the interaction of sebum, perspiration and Corynebacterium minutissimum onto the axillary skin surface.

A non-therapeutic method for cleansing the skin is provided and comprises the step of topically applying onto an area of need of treatment the personal cleansing composition as set out herein, and optionally, removing the composition following its application; preferably wherein the skin is cleaned with mildness.

An area of need of treatment may be a keratinous tissue surface. A keratinous tissue refers to keratin-containing layers positioned as the outermost protective covering of mammals which includes, but is not limited to, skin, hair, nails, or cuticles.

The area of need of treatment may be preferably the human skin surface, or scalp surface.

A method of cleaning the skin while avoiding the harsh effect on the skin of an anionic surfactant comprising or consisting of sodium lauryl sulfate by washing the skin with a personal cleansing composition as set out herein.

A method of preserving a personal cleansing composition is provided and comprises the step of providing a personal cleansing composition and the step of adding in the personal cleansing composition a salt of the alkenyl sulfate ester having an alkenyl chain, wherein the alkenyl chain comprises from 18 to 20 carbon atoms as set out herein; preferably sodium oleyl sulfate.

In the method, the salt of the alkenyl sulfate ester; preferably sodium oleyl sulfate may be present in an amount ranging from about 0.01-5% by weight of the composition; preferably in an amount ranging from about 0.02-3% by weight of the composition; more preferably in an amount ranging from about 0.05-1.5% by weight of the composition; even more preferably in an amount ranging from about 0.1-1% by weight of the composition; most preferably in an amount ranging from about 0.2-0.5% by weight of the composition.

### Test Methods

It is understood that the Test Methods that are disclosed in the Test Methods Section of the present application should be used to determine the respective values of the parameters described and claimed herein.

### Minimum Inhibitory Concentration (MIC)

### In Vitro Minimum Inhibitory Concentration (MIC) Testing

The in vitro Minimum Inhibitory Concentration (MIC) Assay is a technique for determining the lowest concentration of a particular compound needed to prevent growth of microorganism. Different microorganisms were used to evaluate bacterial inhibition benefits for sulfate surfactants (Table 1)

TABLE 1

| Microorganism | Culture Medium | Benefits |
|---|---|---|
| S. aureusATCC6538 | Trypticase soy broth | Personal |
| (Comparative | | hygiene |
| microorganism) | | |
| E. coli ATCC25922 | Trypticase soy broth | Personal |
| | | hygiene |
| Corynebacterium | Trypticase soy broth | Body odor |
| minutissimum | | |
| ATCC23348 | | |
| Cutibacterium acnes | Brain Heart Infusion | Acne |
| SK137 | broth (Anaerobic) | |

Microorganisms are cultured and maintained in different culture medium. Each respective microorganism stock solution is diluted to 10⁵ to 10⁶ CFU/mL in a respective growth medium. 180 μl broth with the respective target microorganism is allocated into 96 well plates.

For instance, Corynebacterium minutissimum ATCC 23348 is cultured on tryptic soy agar (TSA) (Oxoid CM0131) over 24 hours. The single colonies are then transferred into 20 mL Tryptone Soy Broth (Oxoid CM0129B—TSB) and cultured overnight at 200 rpm/min, 37° C. Bacterial cell in broth will be diluted to 10⁵ to 10⁶ CFU/mL in TSB. 180 μL of this bacterium-MHB mix is added to a 96 well flat-bottomed plate (Nunc™ MicroWell™ 96-Well, CAT. No. 167008).

Sodium oleyl sulfate prepared from two different synthesis batches (Ex. 1A and Ex. 1B), sodium octyl sulfate, sodium decyl sulfate, sodium lauryl sulfate were mixed respectively with in Deionized (DI) water. Octadecyl sulfate sodium was mixed in Dimethyl sulfoxide (DMSO).

Two folds serial dilution are applied to each respective active by mixing 100 μl active stock solution (from 1 wt. %) plus 100 μl sterilized DI water or DMSO in 96 well plates (Nunc™ MicroWell™ 96-Well, CAT. No. 167008). For each assay, 0.001-1 wt. % treatment solutions are prepared for each individual active.

Micropipettes are used to transfer 20 μl of actives at different concentration into each well with 180 μl broth with the respective target microorganisms.

The 96 well plates with test microorganisms and test actives are incubated at 37° C.
a) E. coli are incubated for overnight 16 hours to 24 hour.
b) Corynebacterium minutissimum is incubated for two days.
c) Cutibacterium acnes is incubated for two days under anaerobic conditions.

After incubation, microorganism growth is determined in presence of test actives by visual inspection or spectrophotometer by OD600 (absorbance at 600 nm using Tecan M200 Infinite Pro Microplate Reader) reading in comparison with positive control (broth with individual microorganism) and negative control (Broth only). MIC values are presented as parts per million (ppm) for different actives.

### Kill Rate Test in Liquid Test Method

### Suspension Time-Kill Testing

The suspension time-kill test is a method to directly inoculate a liquid test substance with a high concentration of test microorganisms and then determine the percentage of killed over time, normally represented as log CFU reduction. The objective is to measure the biocidal potential of a liquid antimicrobial active or formulation.

### KRT Test Procedures:

E. coli ATCC25922 is streaked on Tryptic Soy Agar (TSA) Plates and cultured overnight. Pick up one colony of E. coli ATCC25922 and subculture bacterial cell in Tryptic Soy Broth (TSB) @ 200 rpm/min, 37° C. for 12-16 hours.
After culturing, 1 mL bacterial cells in broth are pipetted into 2 mL tubes. Samples are centrifuged for 2 mins at 13000 rpm/min. Cell supernatant are discarded, and bacterial cells are resuspended in 1 mL Saline @ 10⁸ to 10⁹ CFU/mL.
1 mL liquid antimicrobial active to be tested under different concentrations is prepared:
20 mM acetate buffers are used to maintain the test solutions at a pH between 4.0 and 5.7.
10 mM phosphate buffer is used to maintain the test solution at a pH around 7.2.
20 mM Tris-HCl buffers are used to maintain the test solutions at a pH between 8.0 and 9.0.
1 mL saline (NaCl 0.9 wt. %) is used to as negative control in the study.
50 μl of 10⁸ to 10⁹ CFU/mL bacterial cells are added into each treatment and saline control. Samples were thoroughly mixed by vertexing 5 seconds.
After incubating 30 minutes, 50 μl liquid from each treatment sample and saline control are pipetted out into for Total Viable Count Vial (NEOGEN). All measurements are done in duplicates.
The Detection Time (DT) for Total Viable Count Vial is measured by Soleris® Next Generation (NEOGEN) system. DT values are averaged for each treatment and control for further calculation. DT time for each sample is converted to Log (viable cell number) by using algorithm generated for E. coli ATCC25922:

$$Log CFU per sample = a * DT + b ( a , slope ; b intercept )$$

Biocidal potential of different actives at different concentrations and at different pHs in a liquid solution is measured by:

$$Log CFU reduction = Log CFU ( Saline - Control ) - Log CFU ( Treatment )$$

### Cylinder Method

Lather can be measured in accordance with the Cylinder Method. Lather volume is measured using a graduated cylinder and a rotating mechanical apparatus. A 1,000 mL graduated cylinder is used which is marked in 10 mL increments, has a height of 14.5 inches at the 1,000 mL mark from the inside of its base, and has a neck at its top fitted for a plastic insert cap (for example, Pyrex No. 2982). Moderately hard water (about 7 gpg or about 120 ppm) is prepared by dissolving 1.14 grams calcium chloride dihydrate and 1.73 grams magnesium chloride hexahydrate into one U.S. gallon distilled water. The water is maintained at between 40.5-43.3° C. (105-110° F.). The graduated cylinder is heated to about the same temperature by flushing with excess tap water at the same temperature for about 15 seconds, then drying its exterior and shaking briefly upside down to dry the interior. 100.0 grams of the moderately hard water at the indicated temperature is weighed directly into the graduated cylinder. The cylinder is clamped in a mechanical rotating device, which clamps the cylinder vertically with an axis of rotation that transects the center of the graduated cylinder. Using a 3- or 4-place metric balance, invert the plastic cap for the graduated cylinder onto the balance pan and weigh 0.500 grams of composition for compositions less than 19% surfactant (weigh 0.250 grams of composition for compositions greater or equal than 19% surfactant) to within 4 milligrams accuracy, using a holder to keep the cap level. Insert the cap into the graduated cylinder neck while being careful that all composition is now in the space in the cylinder interior. For compositions with very low viscosity which will not remain on the cap surface, 500 mg composition can be added directly to the graduated cylinder. Rotate the cylinder for 25 complete revolutions at a rate of about 10 revolutions per 18 seconds to create a lather and stop in a level, vertical position. When the cylinder stops in a vertical position, start a digital stopwatch. Observing the water draining at the bottom, record the time to the nearest second when the water height measures 50 cc, then 60 cc, then 70 cc and so on until at least 90 cc has drained. Measure and record the total height of the foam in the column interior, which is the lather volume. If the top surface of the lather is uneven, the lowest height at which it is possible to see halfway across the graduated cylinder is the lather volume (mL). If the lather is coarse such that a single or only a few foam cells (“bubbles”) reach across the entire cylinder, the height at which at least about 10 foam cells are required to fill the space is the lather volume, also in mL up from the base. When measuring the lather height, bubbles that are larger than about 25.4 mm (1 inch) across at the top surface are considered free air and not lather. The measurement is repeated and at least three results averaged to obtain the lather volume. In a spreadsheet, calculate the lather density at each observed time point as the volume of foam (total height minus water height) divided by the weight of the foam (100.5 grams minus the weight of water observed, using a density of 1.00 g/cc for water). Fit the 3 time points closest to (ideally, also bracketing) 20 seconds to a 2^{nd} order polynomial equation. Solve the equation for the lather density at 20 seconds, which is the lather density of the composition. Multiply the lather volume by the lather density to obtain the lather mass, in grams.

The entire measurement process should take less than about 3 minutes in order to maintain desired temperature.

### Mini-Immersion Method Protocol

This was a two-day, randomized, blinded (subject & instrument operator), forearm mini-immersion study designed to evaluate the irritation potential of various cleanser solutions versus the water only control. All subjects (n=16) completed informed consent, screening and enrollment procedures and qualified subjects were then scheduled for the treatment visit. During the treatment visit, subjects acclimated for 20 minutes in a temperature and humidity-controlled environment (21+/−1° C., 70+/−2° F. & 30-45% Relative Humidity). During acclimation subjects had treatment sites marked on the volar surface of one forearm. On the second day, the other forearm was used and marked accordingly. Upon completion of acclimation, Baseline Corneometer measurements were obtained to record stratum corneum hydration via capacitance.

Once Baseline Corneometer measurements were completed, subjects had open-ended, glass mini-immersion cups strapped to the forearm with elastic bands. Prepared solutions (10% dilution of the test product in DI water and heated to @44° C.) were then be pipetted into each respective cup according to the study randomization. Once all products were dispensed into the cups, a 15-minute timer was started. Once the 15-minute exposure time elapsed, the solutions were pipetted from the cups into a waste receptacle and the cups were removed from each arm. The forearms were rinsed briefly under running tap (temp b/w 35-37.8° C.; 95-100° F.) and gently patted dry. The subject was permitted to take a 10-minute break and then acclimated again in the temperature and humidity-controlled environment for 30 minutes prior to post treatment one corneometer measurements. Once post treatment one measurements were completed, the entire process was repeated until three, 15-minute exposures had elapsed, each followed by a 10-minute subject break, 30-minute acclimation and 40-minutes post treatment evaluations. During the 15-minute exposure periods, subjects were required to sit quietly with their forearms facing upward, resting comfortably on the arms of a chair or table surface to prevent leaks and/or splashing of the solution within the cups.

### NMR Content Method

The NMR Content Method is used to determine the ratio on a molar and weight percent basis of cis/trans isomers of sodium oleyl sulfate with different NMR signals.

In this method, proton NMR spectroscopy is used to analyze a sample of soluble material in organic solvent, and peaks of different ¹H-NMR domains are integrated and compared against an internal standard to determine the ratio of protons and carbons of the isomers.

A flowable solution less than 10 wt. % of a sample is dissolved in a 5 wt. % stock solution of internal standard in an organic solvent. The solution is transferred to a 5 mm NMR glass grade tube and placed in the sample holder (bore) of an NMR instrument. A Bruker NMR device with 600 MHZ field strength equipped with cryo-Prodigy probe using a quantitative proton sequence was used to acquire the data. Appropriate parameters used were as follows: acquisition time (FID length) of 2.7 s, relaxation time of 60 s, 90-degree pulse widths, spectral width of 20 ppm, 32 k points in the FID, and 8 repetition scans used. In the Fourier transform step, exponential apodization is used with 0.3-Hz line broadening, and the spectrum is phased into absorption. A spline baseline correction is used to ensure flat baseline on either side of peaks to be integrated.

The following peak domains are typically used for the content determination and integrated vs the internal standard:
1) The alkenyl CH region for the cis isomer 5.37 ppm-5.32 ppm
2) The alkenyl CH region for the trans isomer 5.40 ppm-5.38 ppm
   Molar ratios and weight percents are calculated as follows:

$$Cis / trans molar ratio = ( inregral cis region ) / ( integral trans region )$$

$$Component weight \% = ( ( mass of Internal Standard in sample ) / ( molecular weight of Internal Standard ) ) * ( ( integral cis or trans region ) / ( integral Internal Standard region ) ) * ( molecular weight of oleyl sulfate ) * ( 1 / ( mass of oleyl sulfate material in sample ) ) * 100$$

### Biodegradability Test Method

The biodegradability of the salt of the alkenyl sulfate ester, preferably sodium oleyl sulfate can be tested according to the following method. The Biodegradability Test Method is based on the Organization for Economic Co-operation and Development (OECD) 301B CO₂ evolution biodegradation test method that screens chemicals for ready biodegradability in an aerobic aqueous medium.

In this test, the test substance is suspended in a phosphate buffered nutrient salts media containing an activated sludge inoculum and the formation of carbon dioxide is measured via a closed-circuit respirometer. The test substance is the sole carbon and energy source and under aerobic conditions microorganisms metabolize organic substances producing CO₂ as the ultimate product. The test system is dosed with a known quantity of carbon and biodegradation is calculated as the percent of theoretical CO₂ formation. The test typically runs for 28 days.

Ready biodegradability is a regulator definition that is used to classify materials that meet the pass criteria as described in the OECD 301B guideline. Specifically, the following pass level of biodegradation, obtained within 28 days, may be regarded as evidence of ready biodegradability: 60% theoretical carbon dioxide (ThCO₂). The pass level has to be reached in a 10-day window within the 28-day period of the test. The 10-day window begins when the degree of biodegradation has reached 10% ThCO₂ and ends 10 days later or at day 28 of the test, whichever comes first.

### EXAMPLES

The following examples further describe the compositions and uses described herein. The examples are given solely for the purpose of illustration and are not to be construed as limitations of the present disclosure, as many variations thereof are possible without departing from the spirit and scope of the disclosure. Where applicable, ingredients are identified by chemical or CTFA name, or otherwise defined below.

### Example 1: Process of Making Sodium Oleyl Sulfate

Sodium oleyl sulfate was prepared from reacting Oleyl Alcohol with Sulfur Trioxide Pyridine Complex according to the process below:

To a 1-Liter, 3-neck round bottom reaction flask equipped with a magnetic stir bar is added 104.7 grams (0.3845 moles) of Technical Grade Oleyl Alcohol (Alfa Aesar product #A18018), 350 mL of Dichloromethane (EMD Millipore product #DX0835) and 2 mL of Pyridine (Sigma Aldrich product #270970). A water cooled condenser is attached to one side neck of the reaction flask with a dry nitrogen gas feed at the top of the condenser leading from a gas bubbler. With mixing at room temperature (21° C.) under nitrogen atmosphere, 65.6 grams (0.404 moles) of 98% Sulfur Trioxide Pyridine Complex (Sigma Aldrich product #S7556) is added. The reaction flask is then equipped with a stopper in the center neck and a thermometer in the other side neck. With mixing under nitrogen atmosphere, the reaction mixture is heated at mild reflux overnight using a heating mantle (thermometer reading of 39-40° C.). The reaction mixture is then allowed to cool to room temperature followed by gravimetric filtration through Whatman Grade 4 filter paper to remove any residual solids. The filtered reaction mixture is concentrated by evaporation of Dichloromethane using a rotary evaporator. To the concentrated reaction product is added a solution of 87.4 grams (0.398 moles) of 24.6 wt. % Sodium Methoxide Solution in Methanol (Sigma Aldrich product #156256) and 750 mL of Methanol (EMD Millipore product #MX0475P) and mixed until all solids dissolve. A small sample is taken and dissolved in DI Water and measured to be pH 10-11 using pH test strip. Additional Sodium Methoxide can be added if needed to reach pH 10-11. This mixture is concentrated by evaporation of Methanol and Pyridine using rotary evaporator. To the concentrated product is added 450 mL of Deionized Water (18.2 megohm-cm filtered water from Millipore water filtration unit), 450 mL of Absolute Ethanol (EMD Millipore product #EX0276) and mixed until all solids dissolved. This solution is transferred to a 2-Liter separatory funnel along with 500 mL of Hexanes (EMD Millipore product #HX0299). The contents of the separatory funnel are shaken well and allowed to stand overnight to separate. The next day, the lower aqueous layer is drained off and concentrated by placing in a vacuum oven under reduced pressure to evaporate solvents. Product is occasionally removed from vacuum oven and mixed, broken and eventually ground into smaller pieces and placed back into vacuum oven to facilitate solvent removal. Once the product is no longer losing appreciable mass, the vacuum oven drying is discontinued and the product is transferred to a jar and sealed for storage.

Final active level of Sodium Oleyl Sulfate product is determined to be 86.6 wt. % by the well-established colorimetric two-phase (water/chloroform) mixed indicator (Dimidium Bromide (Sigma Aldrich product #41785)/Patent Blue VF (Sigma Aldrich product #76357)) titration method using Hyamine 1622 (Sigma Aldrich product #51126) as the cationic titrant (ASTM International standard method D-3049-89 “Standard Test Method for Synthetic Anionic Ingredient by Cationic Titration”).

From Example 1, two different synthesis batches (Ex. 1A, Ex. 1B) have been made: Ex. 1A (white powder) contained a mixture of 51 wt. % of the cis-isomer and 6 wt. % of the trans-isomer. Hence, sodium oleyl sulfate comprises a cis-isomer. Sodium oleyl sulfate comprises a mixture of a cis-isomer and a trans-isomer with a weight ratio cis-isomer to trans-isomer of 8.5:1.

Ex. 1B (tan powder) contained a mixture of 72 wt. % of the cis-isomer and 8 wt. % of the trans-isomer. Hence, sodium oleyl sulfate comprises a cis-isomer. Sodium oleyl sulfate comprises a mixture of a cis-isomer and a trans-isomer with a weight ratio cis-isomer to trans-isomer of 9:1.

### Biodegradability Result

Sodium oleyl sulfate Theoretical Carbon dioxide % (ThCO₂) is 83.46% within 28 days and met the 10-day window requirement thus meeting both requirements for ready biodegradability.

### Minimum Inhibition Concentration Assay

Table 2 shows the minimum inhibition concentration of different anionic sulfate surfactants against known bacteria such as Staphylococcus aureus (for comparison), Escherichia coli, bacteria associated with body odor such as Corynebacterium minutissimum, bacteria associated with acne such as Cutibacterium acne, respectively. Two different lots of sodium oleyl sulfate (Ex. 1A, Ex. 1B) have been tested.

TABLE 2
MIC data for sodium oleyl sulfate and other sulfates

| Minimum | sodium | sodium | sodium | octadecyl | sodium | sodium |
|---|---|---|---|---|---|---|
| Inhibition | octyl | decyl | lauryl | sulfate | oleyl | oleyl |
| Concentration | sulfate | sulfate | sulfate | sodium salt | sulfate | Sulfate |
| MIC (ppm) | (C8) | (C10) | (C12) | (C18) | (Ex. 1A) | (Ex. 1B) |
| Staphylococcus | No effect | No effect | 125 | No effect | 3.9 | 3.9 |
| aureus ATCC6538 | or >1000 | or >1000 | | or >500 | | |
| Escherichia coli | No effect | No effect | No effect | No effect | No effect | No effect |
| ATCC25922 | or >1000 | or >1000 | or >1000 | or >1000 | or >1000 | or >1000 |
| Corynebacterium | No effect | No effect | 125 | No effect | 3.9 | 3.9 |
| minutissimum | or >500 | or >500 | | or >500 | | |
| ATCC 23348 | | | | | | |
| Cutibacterium | No effect | No effect | 31.25 | No effect | 3.9 | 7.8 |
| acne SK137 | or >500 | or >500 | | or >500 | | |
| Cutibacterium | No effect | No effect | 62.5 | No effect | 7.8 | 7.8 |
| acne 2044 NIA | or >500 | or >500 | | or >500 | | |
| (skin isolate) | | | | | | |

*NA: not tested

S. aureus is currently a relevant comparative germ target to inhibit. The results in Table 2 confirms that sodium oleyl sulfate is a known potent antimicrobial agent to inhibit S. aureus. This is to be compared to sodium octadecyl sulfate which differs from sodium oleyl sulfate by the absence of an unsaturation (a double bound within the carbon backbone). Sodium octadecyl sulfate is ineffective against S. aureus. No inhibition have been observed for sodium octyl sulfate and sodium decyl sulfate. Only sodium lauryl sulfate with 12 carbon atoms showed a MIC on S. aureus, however sodium lauryl sulfate is less potent than sodium oleyl sulfate.

The MIC result of sodium oleyl sulfate against gram-positive bacteria such as S. aureus is in the range of potent biocides like benzalkonium chloride.

However, sodium oleyl sulfate was ineffective against gram-negative E. coli at the pH of the assay of 7.2.

Now, sodium oleyl sulfate also demonstrated strong bacterial inhibition effects against Corynebacterium minutissimum and Cutibacterium acne (two strains).

Corynebacterium genus are the primary causal agents of axillary odor, with the key malodor substrates believed to originate from the apocrine gland.

Hence, a personal cleansing composition can be provided for use in a method of treating axillary malodors and killing the growth of Corynebacterium minutissimum onto human skin; wherein the personal cleansing composition comprises a salt of an alkenyl sulfate ester having an alkenyl chain, wherein the alkenyl chain comprises from 18 to 20 carbon atoms, preferably 18 carbon atoms, or an isomer thereof, or mixtures thereof.

C. acne SK137 and C. acne 2044NIA (P&G skin isolate) are from Type IA-2 Clade by whole genome sequencing, which is reported to be associated with skin acne conditions. Also, the data of Table 2 suggests that sodium oleyl sulfate holds the potential to deliver anti-malodor and anti-acne benefits.

It is worth noting that no prior data has been found showing that sodium oleyl sulfate can be used in a method of inhibiting or killing the growth of Cutibacterium acne. Especially, Table 2 shows that the antibacterial potency of sodium oleyl sulfate for inhibiting or killing the growth of Cutibacterium acne over the saturated sodium octadecyl sulfate. Indeed, sodium octadecyl sulfate had no effect on inhibiting or killing the growth of Cutibacterium acne.

In other words, it was not expected by selecting an unsaturated alkyl chain or alkenyl chain, wherein the alkenyl chain comprises from 18 to 20 carbon atoms; preferably 18 carbon atoms, the resulting personal cleansing composition could treat acne.

Hence, a salt of an alkenyl sulfate ester having an alkenyl chain, wherein the alkenyl chain comprises from 18 to 20 carbon atoms; preferably 18 carbon atoms, or an isomer thereof, or mixtures thereof such as sodium oleyl sulfate or a personal cleansing composition as set out herein can be provided for use in a method of inhibiting or killing the growth of bacteria onto human skin; wherein the bacteria is Corynebacterium minutissimum or Cutibacterium acnes.

A personal cleansing composition can be provided for use in a method of treating acne and eradicating Cutibacterium acnes onto a human skin; preferably onto a facial skin; wherein the personal cleansing composition comprises a salt of an alkenyl sulfate ester having an alkenyl chain, wherein the alkenyl chain comprises from 18 to 20 carbon atoms, preferably 18 carbon atoms, or an isomer thereof, or mixtures thereof.

### Killing Rate Testing in Liquid

The previous MIC data on sodium oleyl sulfate was ineffective against gram-negative E. coli at the pH of the assay that was 7.2. However, when setting the pH up to or equal to 4, it was possible to observe some bactericidal activity of sodium oleyl sulfate against E. coli in solution (Table 3).

TABLE 3
Sodium Oleyl sulfate bactericidal activity
against E. coli ATCC25922

| Active &Level | | Sodium Oleyl Sulfate (Ex. 1B) | | |
|---|---|---|---|---|
| (ppm) | | 12.5 | 50 | 200 |
| Test | 4 | 4.3 | 6.6 | 6.6 |
| pH | 5.1 | 0 | <0.3 | <0.3 |

Hence, it appears that a salt of an alkenyl sulfate ester having an alkenyl chain, wherein the alkenyl chain comprises from 18 to 20 carbon atoms; preferably 18 carbon atoms, or an isomer thereof, or mixtures thereof such as sodium oleyl sulfate can have a bactericidal activity against gram-negative bacteria such as E. coli however at a pH up to or equal to 4 in solution.

Hence, a salt of an alkenyl sulfate ester having an alkenyl chain, wherein the alkenyl chain comprises from 18 to 20 carbon atoms; preferably 18 carbon atoms, or an isomer thereof, or mixtures thereof such as sodium oleyl sulfate or a personal cleansing composition as set out herein can be provided for use in a method for killing Escherichia coli at a pH up to or equal to 4 in solution.

### Personal Cleansing Compositions

The following examples were prepared:

### Compositions (wt. %)

| | | C. Ex. A | Ex. B | Ex. C |
|---|---|---|---|---|
| Ingredient | Sodium oleyl sulfate*¹ | 0 | 2 | 9 |
| (wt. %) | Sodium lauryl sulfate*² | 9 | 7 | 0 |
| | Cocamidopropyl betaine*³ | 1 | 1 | 1 |
| | NaCl*⁴ | 1 | 1 | 1 |
| | Kathon CG*⁵ | QS | QS | QS |
| | Citric acid*⁶ | QS | QS | QS |
| Lather | Lather volume/ mL | 563 | 650 | 485 |

Definitions of Components
*¹Sodium oleyl sulfate; P&G
*²Sodium lauryl sulfate; Guangzhou Litze Chemical Co., Ltd
*³Cocamidopropyl Betaine; Guangzhou Tinci Materials Technology Co., Ltd
*⁴Sodium chloride; Sigma
*⁵Kathon CG, Dupont
*⁶Citric acid powder; Yixing Union Biochemical
q.s.: sufficient quantity

### Lather Benefits

Some personal cleansing compositions may perform poorly for lathering and hence cleaning, which are relevant features for consumers. Personal cleansing compositions comprising the salt of an alkenyl sulfate ester having the alkenyl chain, wherein the alkenyl chain comprises from 18 to 20 carbon atoms, as set out herein such as sodium oleyl sulfate can also have consumer acceptable lather properties as shown just above.

Lather can be measured in accordance with the Cylinder Method described herein. Compositions may have a lather volume of about 450 mL or more; preferably about 480 mL or more; more preferably from about 480 mL to about 1050 mL. Compositions may have a lather density of about 0.03 g/cc, about 0.04 g/cc, about 0.05 g/cc, 0.055 g/cc, 0.06 g/cc, 0.065 g/cc, or more. Compositions may have a lather mass of about 20 g, about 25 g, about 30 g, about 35 g, about 40 g, about 45 g, or more.

Hence, Ex. B and Ex. C have acceptable lather properties in terms of lather volume. Notably, Ex. B comprising a mixture of sodium oleyl sulfate with sodium lauryl sulfate shows an increased lather volume.

### Mini-Immersion Corneometer Results

The following examples were prepared and assessed in terms of mildness properties:

### Compositions (wt. %)

| Ingredient (wt. %) | C. Ex. D | Inventive Example E |
|---|---|---|
| Sodium lauryl sulfate | 10% | — |
| Sodium oleyl sulfate | — | 10% |
| Kathon CG | 0.05% | 0.05% |
| Citric acid | pH = 5.5 +/− 0.2 | pH = 5.5 +/− 0.2 |
| | Actual = 5.64 | Actual = 5.62 |
| Water | Q.S. | Q.S. |

Corneometer Results at Baseline before treatment

| | | | Statistical |
|---|---|---|---|
| | Corneometer | Standard | Grouping |
| Code (base size) | Mean | Error | (p < 0.05) |
| C. Ex. D: Sodium lauryl sulfate | 27.078 | 1.654 | a |
| Ex. E: Sodium oleyl sulfate | 27.079 | 1.654 | a |
| Control: Water only | 26.525 | 1.654 | a |

Corneometer Results after treatment ONE (higher is better)

| | | | Statistical |
|---|---|---|---|
| | Corneometer | Standard | Grouping |
| Code | Mean | Error | (p < 0.05) |
| C. Ex. D: Sodium lauryl sulfate | 17.435 | 0.672 | a |
| Ex. E: Sodium oleyl sulfate | 19.508 | 0.672 | b |
| Control: Water only | 22.395 | 0.672 | c |

Corneometer Results after treatment TWO (higher is better)

| | | | Statistical |
|---|---|---|---|
| | Corneometer | Standard | Grouping |
| Code | Mean | Error | (p < 0.05) |
| C. Ex. D: Sodium lauryl sulfate | 16.790 | 0.668 | a |
| Ex. E: Sodium oleyl sulfate | 18.471 | 0.685 | b |
| Control: Water only | 23.193 | 0.667 | c |

Corneometer Results after treatment THREE (higher is better)

| | | | Statistical |
|---|---|---|---|
| | Corneometer | Standard | Grouping |
| Code | Mean | Error | (p < 0.05) |
| C. Ex. D: Sodium lauryl sulfate | 16.254 | 0.750 | a |
| Ex. E: Sodium oleyl sulfate | 19.158 | 0.770 | b |
| Control: Water only | 23.255 | 0.750 | c |

The results show that a personal cleansing composition comprising sodium oleyl sulfate was significant milder than a personal cleansing composition comprising sodium lauryl sulfate based on higher corneometer results of Ex. E vs. C. Ex. D after treatment one, two, and three.

Thus, the personal cleansing composition as set out herein can be used for cleaning skin with mildness; compared to a control composition comprising 10% of sodium lauryl sulfate by weight of the control composition, 0.05% of kathon CG by weight of the control composition, q.s. citric acid at pH of 5.5±0.2 in q.s. water.

### Additional Examples/Combinations

A. A salt of an alkenyl sulfate ester having an alkenyl chain, wherein the alkenyl chain comprises from 18 to 20 carbon atoms, or an isomer thereof, or mixtures thereof; preferably wherein the salt of the alkenyl sulfate ester having the alkenyl chain comprises a sulfate ester group and has the formula (I) as follows:

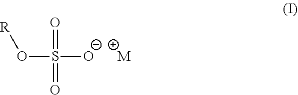

wherein R is an alkenyl chain and the alkenyl chain comprises from 18 to 20 carbon atoms and wherein the salt (M) of the alkenyl sulfate ester is selected from the group consisting of an alkali metal, ammonium, and alkanolamine; more preferably sodium oleyl sulfate for use in a method of inhibiting or killing the growth of bacteria onto human skin, wherein the bacteria is selected from the group consisting of Escherichia coli at a pH up to or equal to 4 in solution, Cutibacterium acnes, and Corynebacterium minutissimum.
B. A salt of an alkenyl sulfate ester having an alkenyl chain, wherein the alkenyl chain comprises from 18 to 20 carbon atoms, or an isomer thereof, or mixtures thereof; preferably sodium oleyl sulfate for use in a method of eradicating Cutibacterium acnes onto a human skin; more preferably onto a facial skin.
C. A salt of an alkenyl sulfate ester having an alkenyl chain, wherein the alkenyl chain comprises from 18 to 20 carbon atoms, or an isomer thereof, or mixtures thereof; preferably sodium oleyl sulfate for use in a method of treating axillary malodors and killing the growth of bacteria onto human skin; wherein the bacteria is Corynebacterium minutissimum.
D. A personal cleansing composition comprising the salt of an alkenyl sulfate ester as set out herein for use in a method of inhibiting or killing the growth of bacteria onto human skin, wherein the bacteria is selected from the group consisting of Escherichia coli at a pH up to or equal to 4 in solution, Cutibacterium acnes, and Corynebacterium minutissimum.
E. A personal cleansing composition for use in a method of treating acne and eradicating Cutibacterium acnes onto a human skin; preferably onto a facial skin; wherein the personal cleansing composition comprises a salt of an alkenyl sulfate ester having an alkenyl chain, wherein the alkenyl chain comprises from 18 to 20 carbon atoms, preferably 18 carbon atoms, or an isomer thereof, or mixtures thereof.
F. A personal cleansing composition for use in a method of treating axillary malodors and killing the growth of bacteria onto human skin; wherein the bacteria is Corynebacterium minutissimum, wherein the personal cleansing composition comprises a salt of an alkenyl sulfate ester having an alkenyl chain, wherein the alkenyl chain comprises from 18 to 20 carbon atoms, preferably 18 carbon atoms, or an isomer thereof, or mixtures thereof.
G. Non-therapeutical use of a salt of an alkenyl sulfate ester having an alkenyl chain, wherein the alkenyl chain comprises from 18 to 20 carbon atoms as set out herein; preferably sodium oleyl sulfate, as a malodor counteracting agent on products selected from the group consisting of consumer products, household products, cosmetic and personal care products, products for use on the human body, products applied to the human skin, and perfumed consumer goods.
H. The non-therapeutic use of Paragraph G. wherein the salt of the alkenyl sulfate ester having an alkenyl chain, wherein the alkenyl chain comprises from 18 to 20 carbon atoms as set out herein; preferably sodium oleyl sulfate is used as a malodor counteracting agent in a personal cleansing composition onto the axillary skin surface.
I. Non-therapeutic use of the personal cleansing composition as set out herein for providing a cleansing benefit.
J. Non-therapeutic use of the personal cleansing composition as set out herein for improving hand hygiene.
K. Non-therapeutic use of the personal cleansing composition as set out herein for its mildness towards the skin; preferably for preventing dryness of the skin.
L. Non-therapeutic use of a salt of an alkenyl sulfate ester having an alkenyl chain, wherein the alkenyl chain comprises from 18 to 20 carbon atoms, or an isomer thereof, or mixtures thereof; preferably sodium oleyl sulfate in a personal cleansing or personal cleansing composition to increase the mildness to the skin of the composition; more preferably by preventing skin tightness, skin roughness and/or skin irritation.
M. Non-therapeutic use of a personal cleansing composition for cleaning skin with mildness; preferably compared to a control composition comprising 10% of sodium lauryl sulfate, 0.05% of kathon CG, q.s. citric acid at pH of 5.5±0.2 in q.s. water; wherein the personal cleansing composition comprises a salt of an alkenyl sulfate ester having an alkenyl chain, wherein the alkenyl chain comprises from 18 to 20 carbon atoms, preferably 18 carbon atoms, or an isomer thereof, or mixtures thereof.
N. Non-therapeutic use of a personal cleansing composition for preventing dryness of the skin; wherein the personal cleansing composition comprises a salt of an alkenyl sulfate ester having an alkenyl chain, wherein the alkenyl chain comprises from 18 to 20 carbon atoms, preferably 18 carbon atoms, or an isomer thereof, or mixtures thereof.
O. A method of inhibiting or killing the growth of bacteria on an area of need of treatment; wherein the bacteria is Escherichia coli at a pH up to or equal to 4, Corynebacterium minutissimum, and/or Cutibacterium acnes, comprising the step of topically applying onto an area of need of treatment an effective amount of a salt of an alkenyl sulfate ester as described herein, and optionally, removing the composition following its application.
P. A method of killing a bacteria on a skin surface; wherein the bacteria is Escherichia coli at a pH up to or equal to 4, Corynebacterium minutissimum, and/or Cutibacterium acnes, comprising the step of topically applying onto an area of need of treatment an effective amount of a personal cleansing composition as set out herein, and optionally, removing the composition following its application.
Q. A method of controlling or eradicating Cutibacterium acnes from a user's skin comprising the step of applying an effective amount of a personal cleansing composition as set out herein onto a desired skin surface of a user in need thereof; preferably onto a facial skin surface.
R. The method of Paragraph Q, for reducing or eliminating acne on skin.
S. A method of controlling or eradicating Corynebacterium minutissimum from an axillary skin surface comprising the step of applying an effective amount of a personal cleansing composition as set out herein onto the desired axillary skin surface of a user in need thereof; preferably onto the axillary skin surface.
T. The method of Paragraph S, for minimizing malodors or unpleasant odors caused by the interaction of sebum, perspiration and Corynebacterium minutissimum onto the axillary skin surface.
U. A non-therapeutic method for cleansing the skin, wherein the method comprises the step of topically applying onto an area of need of treatment the personal cleansing composition as set out herein, and optionally, removing the composition following its application; preferably wherein the skin is cleaned with mildness.
V. A method of cleaning the skin while avoiding the harsh effect on the skin of an anionic surfactant consisting of sodium lauryl sulfate by washing the skin with a personal cleansing composition as set out herein.
W. Use of a personal cleansing composition comprising the salt of an alkenyl sulfate ester as set out herein for the manufacture of a medicament for inhibiting or killing the growth of bacteria onto human skin, wherein the bacteria is selected from the group consisting of Escherichia coli at a pH up to or equal to 4 in solution, Cutibacterium acnes, and Corynebacterium minutissimum.
X. Use of a personal cleansing composition for the manufacture of a medicament for treating acne and eradicating Cutibacterium acnes onto a human skin; preferably onto a facial skin; wherein the personal cleansing composition comprises a salt of an alkenyl sulfate ester having an alkenyl chain, wherein the alkenyl chain comprises from 18 to 20 carbon atoms, preferably 18 carbon atoms, or an isomer thereof, or mixtures thereof.
Y. Use of a personal cleansing composition for the manufacture of a medicament for treating axillary malodors and killing the growth of bacteria onto human skin; wherein the bacteria is Corynebacterium minutissimum, wherein the personal cleansing composition comprises a salt of an alkenyl sulfate ester having an alkenyl chain, wherein the alkenyl chain comprises from 18 to 20 carbon atoms, preferably 18 carbon atoms, or an isomer thereof, or mixtures thereof.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of inhibiting or killing the growth of bacteria onto human skin, wherein the bacteria comprises at least one of Escherichia coli at a pH up to or equal to about 4 in solution, Cutibacterium acnes, Corynebacterium minutissimum, or a combination thereof, comprising a step of topically applying onto human skin an effective amount of a personal cleansing composition; wherein the personal cleansing composition comprises a salt of an alkenyl sulfate ester having an alkenyl chain, wherein the alkenyl chain comprises from 18 to 20 carbon atoms, or an isomer thereof, or mixtures thereof.

2. The method of claim 1, wherein the salt of the alkenyl sulfate ester having the alkenyl chain comprises a sulfate ester group and has the formula (I) as follows: 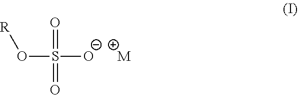
wherein R is an alkenyl chain and the alkenyl chain comprises from 18 to 20 carbon atoms; or an isomer thereof, or mixtures thereof.

3. The method of claim 2, wherein the salt of the alkenyl sulfate ester is selected from the group consisting of an alkali metal, ammonium, and alkanolamine.

4. The method of claim 1, wherein the salt of the alkenyl sulfate ester having the alkenyl chain is present in an amount ranging from about 0.01-50% w/w; alternatively wherein the salt of the alkenyl sulfate ester is present in an amount ranging from about 0.01-5% w/w.

5. The method of claim 1, wherein the salt of the alkenyl sulfate ester having the alkenyl chain is chosen from sodium oleyl sulfate, TEA-oleyl sulfate, ammonium oleyl sulfate, sodium elaidyl sulfate, sodium petroselinyl sulfate, sodium linoleyl sulfate, sodium linolenyl sulfate, sodium gadoleyl sulfate, or combinations thereof.

6. The method of claim 5, wherein the salt of the alkenyl sulfate ester is chosen from sodium oleyl sulfate, TEA-oleyl sulfate, ammonium oleyl sulfate or combinations thereof.

7. The method of claim 6, wherein the salt of the alkenyl sulfate ester comprises sodium oleyl sulfate or ammonium oleyl sulfate; optionally wherein the salt of the alkenyl sulfate ester is sodium oleyl sulfate.

8. The method of claim 7, wherein sodium oleyl sulfate is present in an amount ranging from about 0.01-50% w/w; alternatively wherein sodium oleyl sulfate is present in an amount ranging from about 0.01-5% w/w.

9. The method of claim 7, wherein sodium oleyl sulfate comprises a cis-isomer.

10. The method of claim 9, wherein sodium oleyl sulfate comprises a mixture of a cis-isomer and a trans-isomer with a weight ratio cis-isomer to trans-isomer from about 8:1 to about 99:1;

11. The method of claim 10, wherein the weight ratio cis-isomer to trans-isomer from about or from about 8.5:1 to about 95:1.

12. The method of claim 11, weight ratio cis-isomer to trans-isomer from about or from about 8.5:1 to about 9:1.

13. The method of claim 1, wherein the composition is in the form of a topical personal cleansing composition, wherein the composition further comprises a dermatologically acceptable carrier for the personal cleansing composition.

14. The method of claim 1, wherein the composition wherein the bacteria is Corynebacterium minutissimum; wherein the human skin is axillary skin; and the method treats axillary malodors.

15. The method of claim 14, wherein the method further minimizes malodors or unpleasant odors caused by the interaction of sebum, perspiration and Corynebacterium minutissimum onto an axillary skin surface.

16. A method of treating acne and eradicating Cutibacterium acnes onto a human skin; comprising the step of topically applying onto human skin an effective amount of a personal cleansing composition; wherein the personal cleansing composition comprises a salt of an alkenyl sulfate ester having an alkenyl chain, wherein the alkenyl chain comprises from 18 to 20 carbon atoms, or an isomer thereof, or mixtures thereof.

17. The method of claim 16, wherein the human skin is facial skin.
